(19) United States
(12) Patent Application Publication
(10) Pub. No.: US 2002/0002167 A1
(43) Pub. Date: Jan. 3, 2002

(54) Dihydropyridine compounds and composition containing the same

(75) Inventors: Seiji Niwa, Kawasaki-shi (JP); Seiji Ohno, Kawasaki-shi (JP); Akira Takahara, Kawasaki-shi (JP); Morikazu Kito, Kawasaki-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: AJINOMOTO CO., INC., Chuo-ku (JP)

(21) Appl. No.: 09/839,214

(22) Filed: Apr. 23, 2001

Related U.S. Application Data
(63) Continuation of application No. PCT/JP99/05840, filed on Oct. 22, 1999.

(30) Foreign Application Priority Data
Oct. 23, 1998 (JP) 10-303066

(51) Int. Cl. C07D 413/02; C 07D 4 3/02; C 07D 4 1/02; A61K 31/5377; A61K 31/497; A61K 31/496; A61K 31/506; A61K 31/501

(52) U.S. Cl. 514/252.03; 514/235.5; 514/253.13; 514/255.05; 514/256; 514/314; 514/318; 544/333; 544/360; 546/193; 544/405; 544/131

(57) ABSTRACT

A dihydropyridine compound of the formula:

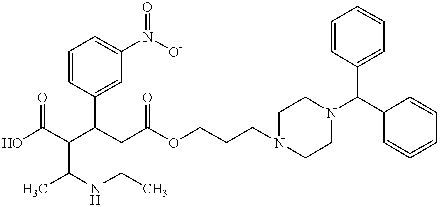

or a pharmaceutically acceptable salt thereof is provided. These compounds are useful as N-type calcium channel antagonists, particularly in therapeutic agents and/or compositions for various diseases, such as acute stage of ischemic cerebrovascular disorders caused by cerebral infarction or intracerebral bleeding, and Alzheimer's disease.

### BACKGROUND OF THE INVENTION

1. Field of the Invention

The present invention relates to certain dihydropyridine compounds, a composition containing the same, and the use thereof as a medicine. The activation of N-type calcium channel correlates with various diseases, for example, acute stage of ischemic cerebrovascular disorders caused by cerebral infarction or intracerebral bleeding (including subarachnoidal hemorrhage); progressive neurodegenerative diseases such as Alzheimer's disease, AIDS related dementia and Parkinson's disease, dementia due to cerebrovascular disorder and ALS; neuropathy caused by head injury; various pains such as pain caused by spinal injury, diabetes or thromboangiitis obliterans, postoperative pain, migraine and visceral pain; various diseases associated with psychogenic stress such as bronchial asthma, unstable angina and irritable colitis; emotional disorder and withdrawal symptoms after addiction to drugs such as ethanol withdrawal symptoms. The compounds of the present invention inhibit activation of the N-type calcium channel and, therefore, are useful in the treatment of, and the remedies for these diseases.

2. Description of the Background

Calcium channels are now classified into subtypes of L, N, P, Q, R and T. Each subtype of calcium channel is organ-specifically distributed. It is known that, in particular, N-type calcium channel is widely distributed in pars centralis, peripheral nerves and adrenomedullary cells and participates in neuronal cell death, regulation of blood catecholamine level and control of senses, such as perception.

It has been confirmed that omega conotoxin GVIA and omega conotoxin MVIIA, which are peptides selectively inhibiting N-type calcium channel, inhibit the release of excitatory neurotransmitters in sliced brain preparation. It is also confirmed in animal experiments that they inhibit the progress of neuronal necrosis associated with cerebrovascular disorders. It is generally considered that compounds having N-type calcium channel blocking action are clinically effective in the treatment of acute stage of ischemic cerebrovascular disorders caused by cerebral infarction or intracerebral bleeding (including subarachnoidal hemorrhage); progressive neurodegenerative diseases such as Alzheimer's disease, AIDS related dementia and Parkinson's disease, dementia due to cerebrovascular disorder and ALS; and neuropathy caused by head injury. Further, it has been confirmed in animal tests that omega conotoxin MVIIA relieves pain induced by formaldehyde, hot plate and peripheral neuropathy. Accordingly, omega conotoxin MVIIA is considered to be clinically effective against various pains, such as pain caused by spinal injury, diabetes or thromboangiitis obliterans, postoperative pain, migraine and visceral pain. In addition, because omega conotoxin GVIA inhibits the release of catecholamine from cultured sympathetic ganglion cells, catecholamine secretion from canine adrenal medulla and the contraction of the isolated blood vessel by electric stimulation of the perivascular nerve, it is considered that compounds having N-type calcium channel-blocking effects are clinically effective against various diseases related to psychogenic stress such as bronchial asthma, unstable angina and irritable colitis. Neuropharmacol., 32, 1141 (1993).

Some peptidergic and non-peptidergic compounds which selectively affect N-type calcium channels have been disclosed. See, for example, WO 9313128. However, none of these compounds have ever actually been used clinically as a medicine. Some of the compounds which affect N-type calcium channels are also effective against various types of calcium channels of other than N-type. See British Journal of Pharmacology, 122 (1) 37-42, 1997. For example, compounds having an antagonistic effect on L-type calcium channels which are very closely related to hypotensive effect, cannot be used for diseases for which N-type antagonists will be used, such as cerebral stroke, neuralgia, terminal cancer pain and pain of spinal injury.

Hence, a need exists for compounds having a selective antagonistic effect on N-type calcium channels.

### SUMMARY OF THE INVENTION

Accordingly, it is an object of the present invention is to provide compounds having a selective antagonistic effect on N-type calcium channels.

It is another object of the present invention is to provide antagonists to N-type calcium channel.

Moreover, it is yet another object of the present invention is to provide a therapeutic composition for any of acute stage of ischemic cerebrovascular disorders caused by cerebral infarction or intracerebral bleeding, Alzheimer's disease, AIDS related dementia, Parkinson's disease, progressive neurodegenerative diseases, neuropathy caused by head injury, pain caused by thromboangiitis obliterans, postoperative pain, migraine, visceral pain, bronchial asthma, unstable angina, irritable colitis and withdrawal symptoms due to drug addiction.

The above objects and others are provided, in part, by compounds of the formula (1) as described herein.

### DETAILED DESCRIPTION OF THE PREFERRED EMBODIMENTS

The present invention provides dihydropyridine compounds of the following formula (1) and pharmaceutically acceptable salts thereof.

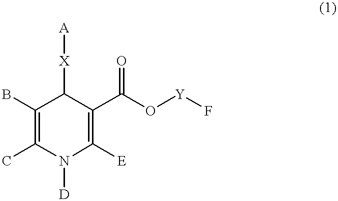

wherein A represents a group of the following general formula (2), or 1-naphthyl, 2-naphthyl, thiophene-3-yl, thiophene-2-yl, furan-3-yl, furan-2-yl, pyridine-4-yl, pyridine-3-yl, pyridine-2-yl, indole-2-yl, indole-3-yl, quinoline-2-yl, quinoline-3-yl, quinoline-4-yl, quinoline-5-yl, quinoline-6-yl, quinoline-7-yl, quinoline-8-yl, cyclohexyl or cyclopentyl group:

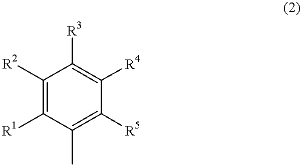

wherein R¹, R², R³, R⁴ and R⁵ are the same or different from each other and each represents a hydrogen atom, halogen atom, hydroxyl group, carboxyl group, amino group, cyano group, nitro group, a lower alkyl group, a lower alkoxyl group, a lower alkenyl group, a lower alkynyl group, a lower alkylamino group, a lower alkylthio group, a lower alkanoyl group, a hydroxy-lower alkyl group, a hydroxy-lower alkoxyl group, a hydroxy-lower alkenyl group, a halogeno-lower alkyl group, a halogeno-lower alkoxyl group, a halogeno-lower alkenyl group, an aryl-lower alkoxyl group, a lower-alkoxycarbonyl group, an aryl group, a heteroaryl group or an aroyl group;

B represents carbamoyl group, cyano group, nitro group, acetyl group or carboxyl group;

C represents a hydrogen atom, methyl group, ethyl group or dimethoxymethyl group;

D represents a hydrogen atom, a lower alkyl group, a hydroxy-lower alkyl group or an aryl-lower alkyl group;

E represents a hydrogen atom, methyl group, ethyl group, dimethoxymethyl group or cyano group;

F represents a heterocyclic group or a cycloalkyl group;

X represents an interatomic bond, —CH₂—, —CH₂CH₂—, —CH═CH— or —C═C—; and

Y represents an interatomic bond, —CH₂— or a group of any of the following formulae (3) to (15):

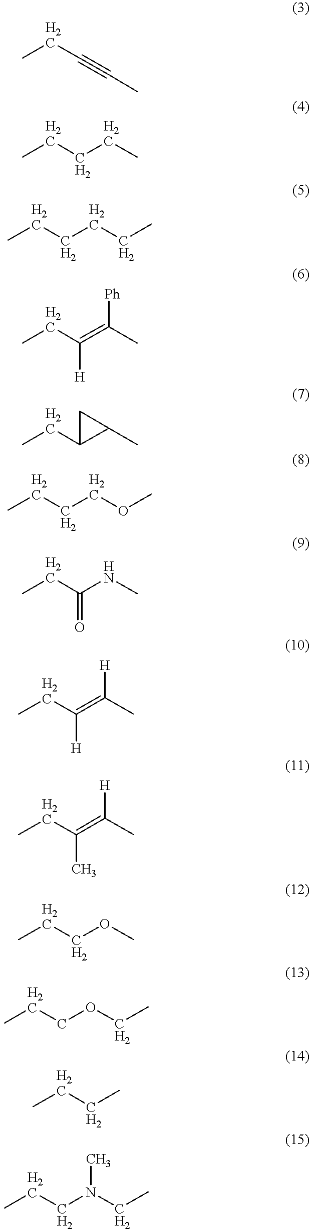

with the proviso that when Y represents any of the groups of formulae (3) to (15), the heterocyclic groups represented by F exclude groups of the formula (16), cyclohexyl group, thiophene-3-yl group, thiophene-2-yl group, furan-3-yl group, furan-2-yl group, pyridine-4-yl group, pyridine-3-yl group and pyridine-2-yl group:

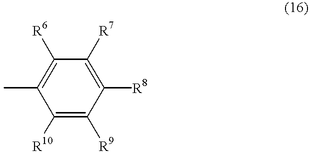

wherein R⁶, R⁷, R⁸, R⁹ and R¹⁰ are the same or different from each other, and each represents hydrogen atom, a halogen atom, hydroxyl group, carboxyl group, amino group, cyano group, nitro group, a lower alkyl group, a lower alkoxyl group, a lower alkenyl group, a lower alkynyl group, a lower alkylamino group, a lower alkylthio group, a lower alkanoyl group, a hydroxy-lower alkyl group, a hydroxy-lower alkoxyl group, a hydroxy-lower alkenyl group, a halogeno-lower alkyl group, a halogeno-lower alkoxyl group, a halogeno-lower alkenyl group, an aryl-lower alkoxyl group, a lower-alkoxycarbonyl group or an aroyl group, and two of R¹ through R³ in general formula (2) may be bonded to each other to form a ring.

The present invention also provides an N-type calcium channel antagonist containing one or more dihydropyridine compounds of above formula (1) or pharmaceutically acceptable salts thereof as an active ingredient.

The present invention further provides a therapeutic agent containing one or more of the above-described dihydropyridine compounds or pharmaceutically acceptable salts thereof as the active ingredient, for any of acute stage of ischemic cerebrovascular disorders caused by cerebral infarction or intracerebral bleeding, Alzheimer's disease, AIDS related dementia, Parkinson's disease, progressive neurodegenerative diseases, dementia due to cerebrovascular disorder, pain caused by thromboangiitis obliterans, postoperative pain, migraine, visceral pain, bronchial asthma, unstable angina, irritable colitis and withdrawal symptoms after addiction to drugs.

The present invention also provides a pharmaceutical composition containing one or more of the dihydropyridine compounds or the pharmaceutically acceptable salts thereof and a carrier and/or a diluent.

The term “lower” as used herein indicates that the group has 1 to 6 carbon atoms. Alkyl groups, themselves, and also alkyl groups in alkoxyl groups, alkenyl groups, alkylamino groups, alkylthio groups and alkanoyl groups may be either linear or branched. Examples of these alkyl groups are methyl group, ethyl group, propyl group, isopropyl group, butyl group and secondary and tertiary butyl groups. Among them, those having 1 to 3 carbon atoms are preferred. The aryl-lower alkoxyl groups include, for example, benzyloxy group. The halogen atoms include fluorine, chlorine, bromine and iodine atoms. The aryl groups are both substituted and unsubstituted aryl groups. They are preferably phenyl group and substituted phenyl group, and the substituents are particularly halogens, alkyl groups and alkoxyl groups. Examples of the aroyl groups include benzoyl group and pyridylcarbonyl group.

The heterocyclic groups in the present invention may have a substituent. The substituents are, for example, halogen atoms, alkyl groups, alkanoyl groups, aryl groups, arylalkyl groups, alkoxyl groups, nitro group and cyano group.

The heterocyclic group F in the compounds of the formula (1) of the present invention is preferably pyrrolidine, piperazine, pyrazolidine, imidazolidine, tetrahydrofuran, tetrahydropyran, dioxane, tetrahydrothiophene, morpholine, imidazole, pyrrolidinone, oxazole, isoxazole, pyrimidine, pyrazine, pyridazine or piperidine group.

R¹, R², R³, R⁴ and R⁵ in the general formula (2), which are the same or different from each other, are preferably hydrogen atom, a halogen atom, hydroxyl group, carboxyl group, cyano group, nitro group, a lower alkyl group, a lower alkoxyl group, a halogeno-lower alkyl group or a lower-alkoxycarbonyl group. Preferably D represents hydrogen atom, X represents an interatomic bond, Y represents an interatomic bond, methylene group, ethylene group or propylene group, and B represents carboxyl group.

It is more preferred that R¹, R², R³, R⁴ and R⁵ in the formula (2), which are the same or different from each other, are hydrogen atom, a halogen atom, carboxyl group, cyano group, nitro group or a halogeno-lower alkyl group, C represents methyl group, E represents methyl group and F represents any of pyrrolidine group, piperazine group, imidazole group, pyrrolidinone group and piperidine group.

It is more preferred that A is represented by the formula (2), wherein R¹, R³, R⁴ and R⁵ each represent hydrogen atom and R² represents nitro group, C represents methyl group, E represents methyl group and F represents piperidine group or piperazine group.

It is particularly preferred that A is represented by the formula (2), wherein R¹, R³, R⁴ and R⁵ each represent hydrogen atom and R² represents nitro group, B represents carboxyl group, C represents methyl group, D represents hydrogen atom, E represents methyl group, F represents piperidine group or piperazine group, X represents an interatomic bond, and Y represents any of interatomic bond, methylene group, ethylene group and propylene group.

Dihydropyridine compounds (1) of the present invention can be produced by processes described below:

For example, dihydropyridine derivatives (1-1) wherein D represents hydrogen atom and B represents carboxyl group can be produced by the following reaction scheme:

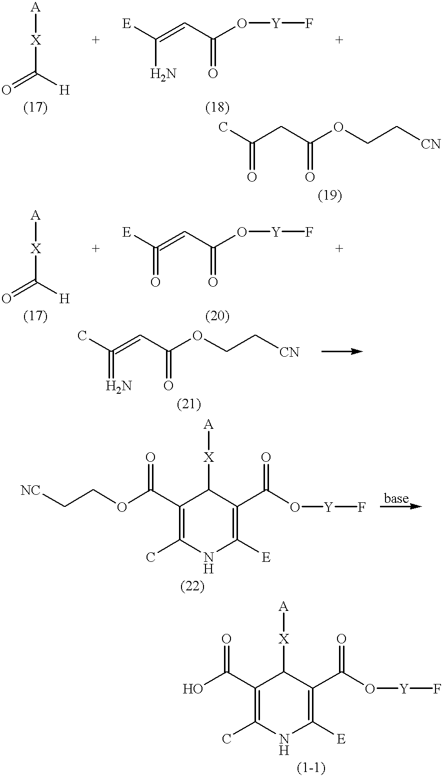

wherein A, C, E, F, X and Y are as defined above.

Namely, a dihydropyridinedicarboxylic acid diester (22) can be obtained by reacting an aldehyde (17) with a 3-aminocrotonic acid ester (18) having a substituent E at the 3-position and 2-cyanoethyl ester of ketocarboxylic acid (19) or by reacting the aldehyde (17) with a ketoester (20) and 2-cyanoethyl ester of 3-aminocrotonic acid (21) having a C substituent at the 3-position. The dihydropyridinecarboxylic acid compounds (1-1) of the present invention can be produced by treating the obtained diester of dihydropyridinedicarboxylic acid with a base such as sodium hydroxide.

In another process, dihydropyridinecarboxylic acid derivatives (1-1) of the present invention can be produced by the following reaction scheme:

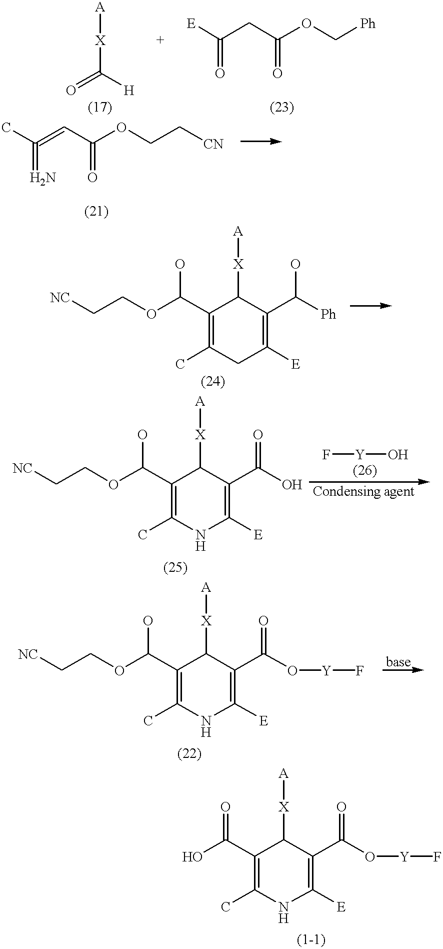

Namely, cyanoethyl esters of benzyl dihydropyridinedicarboxylates (24) can be obtained by reacting the aldehyde (17) with a benzyl ketocarboxylate (23) and 2-cyanoethyl 3-aminocrotonate having a C substituent at the 3-position. Monocyanoethyl dihydropyridinedicarboxylates (25) can be obtained by hydrogenating the obtained esters (24) in ethyl acetate in the presence of palladium catalyst. Dihydropyridinedicarboxylic acid diesters (22) can be obtained by reacting the obtained compound (25) with an alcohol (26) in the presence of a condensing agent such as WSC. Dihydropyridinecarboxylic acid derivatives (1-1) of the present invention can be produced by treating the obtained dihydropyridinedicarboxylic diesters (22) with a base such as sodium hydroxide.

Dihydropyridine compounds (1-2) of the above formula wherein B represents carbamoyl group, nitro group or acetyl group can be produced by reacting acetoacetic acid amide (27), nitroacetone (28) or acetylacetone (29) with, the aldehyde (17) and the 3-aminocrotonic acid ester (18) by the following reaction scheme:

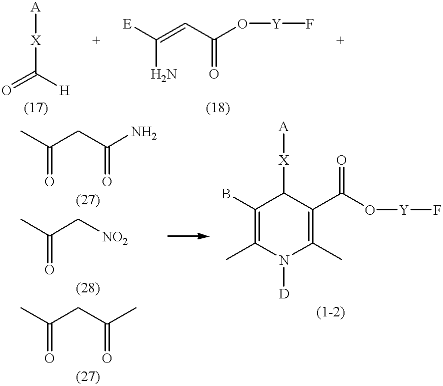

wherein A, D, F, X and Y are as defined above.

Dihydropyridine compounds (1-3) of the above formula wherein B represents cyano group can be produced by reacting the aldehyde (17) with the acetoacetic acid ester (20) and 3-aminocrotonitrile (30) by the following reaction scheme:

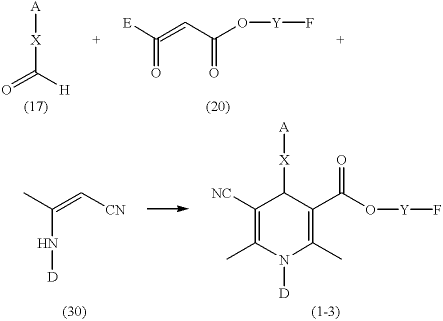

wherein A, D, F, X and Y are as defined above.

When the compounds of the formula (1) can form salts thereof, the salts are pharmaceutically acceptable ones such as ammonium salts, salts thereof with alkali metals, e.g. sodium and potassium, salts thereof with alkaline earth metals, e.g. calcium and magnesium, salts thereof with aluminum and zinc, salts thereof with organic amines, e.g. morpholine and piperidine, and salts thereof with basic amino acids, e.g. arginine and lysine.

The compounds of the formula (1) or salts thereof are administered as they are or in the form of various medicinal compositions to patients. The dosage forms of the medicinal compositions are, for example, tablets, powders, pills, granules, capsules, suppositories, solutions, sugar-coated tablets and depots. They can be prepared with ordinary preparation assistants such as carriers and diluents by conventional methods. For example, the tablets may be prepared by mixing one or more of the dihydropyridine compounds, the active ingredients of the present invention, with any known adjuvants such as inert diluents, e.g. lactose, calcium carbonate and calcium phosphate; binders, e.g. acacia, corn starch and gelatin; extending agents, e.g. alginic acid, corn starch and pre-gelatinized starch; sweetening agents, e.g. sucrose, lactose and saccharin; corrigents, e.g. peppermint, and cherry; and lubricants, e.g. magnesium stearate, talc and carboxymethyl cellulose.

The N-type calcium channel inhibitor containing one or more of the compounds of the formula (1) or the salts thereof as an active ingredient is useful as a remedy in the treatment of various diseases, for example, acute stage of ischemic cerebrovascular disorders caused by cerebral infarction or intracerebral bleeding (including subarachnoidal hemorrhage); progressive neurodegenerative diseases such as Alzheimer's disease, AIDS related dementia and Parkinson's disease, dementia due to cerebrovascular disorder and ALS; neuropathy caused by head injury; pain caused by spinal injury, diabetes or thromboangiitis obliterans, postoperative pain, migraine and visceral pain; various diseases caused by psychogenic stress such as bronchial asthma, unstable angina and irritable colitis; emotional disorder withdrawal symptoms after addiction to drugs such as ethanol withdrawal symptoms.

The dose of the compound or compounds of the formula (1) or salts thereof used for the above-described purpose varies depending on the intended therapeutic effect, administration method, period of the treatment, and age and body weight of the patient. The dose is generally 1 μg to 5 g a day for adults for oral administration, and 0.01 μg to 1 g a day for adults for parenteral administration. Such doses may be administered as simple unit or multiple unit doses

The present invention will now be further illustrated by reference to certain Examples which are provided solely for purposes of illustration and are not intended to be limitative.

### EXAMPLE 1

### Mono(2-(4-benzhydrylpiperazine-1-yl)ethyl)2,6-dimethyl-4-(3-nitrophenyl)-1,4-dihydropyridine-3,5-dicarboxylate

1) Synthesis of 3-(2-(4-benzhydrylpiperazine-1-yl)ethyl)5-(2-cyanoethyl)2,6-dimethyl-4-(3-nitrophenyl)-1,4-dihydropyridine-3,5-dicarboxylate:

774 mg (2.03 mmol) of (2-(4-benzhydrylpiperazine-1-yl)ethyl) acetoacetate, 312 mg (2.03 mmol) of 2-cyanoethyl 3-aminocrotonate and 310 mg (2.05 mmol) of 3-nitrobenzaldehyde were heated at 80° C. under stirring in 20 ml of 2-propanol overnight. 2-Propanol was evaporated under reduced pressure, and the residue was purified by silica gel chromatography (hexane/ethyl acetate: 2/1) to obtain the title compound.

Yield: 695 mg (1.07 mmol) (52.7%)

MS (ESI, m/z) 650 (M+H)⁺

¹H-NMR (CDCl₃):2.37 (3H, s), 2.38 (3H, s), 2.24-2.52 (8H, m), 2.56-2.66 (4H, m), 4.08-4.32 (5H, m), 5.08 (1H, s), 5.84 (1H, s), 7.14-7.44 (11H, m), 7.68 (1H, d), 7.96-7.99 (1H, m), 8.08 (1H, t)

2) Synthesis of mono(2-(4-benzhydrylpiperazine-1-yl)ethyl)2,6-dimethyl-4-(3-nitrophenyl)-1,4-dihydropyridine-3,5-dicarboxylate:

687 mg (1.06 mmol) of 3-(2-(4-benzhydrylpiperazine-1-yl)ethyl) 5-(2-cyanoethyl) 2,6-dimethyl-4-(3-nitrophenyl)-1,4-dihydropyridine-3,5-dicarboxylate was dissolved in 20 ml of methanol. 2.1 ml of 1 N aqueous sodium hydroxide solution was added to the obtained solution, and they were stirred at room temperature for 5.5 hours. 2 N hydrochloric acid was added to the obtained mixture, and then methanol was evaporated under reduced pressure. Water was added to the residue, and the resultant solid was taken by the filtration, washed with water and then with a mixture of hexane and ethyl acetate (3:1), and dried under reduced pressure to obtain the title compound.

Yield: 407 mg (0.68 mmol) (64.3%)

MS (ESI, m/z) 597 (M+H)⁺

¹H-NMR (DMSO-d₆):2.26 (3H, s), 2.30 (3H, s), 2.10-2.65 (10H, m), 3.98-4.34 (3H, m), 4.98 (1H, s), 7.16-7.26 (2H, m), 7.28-7.51 (9H, m), 7.62 (1H, d), 7.91 (1H, d), 7.96 (1H, s), 8.99 (1H, s)

The structural formula of the compound obtained in Example 1 was as follows:

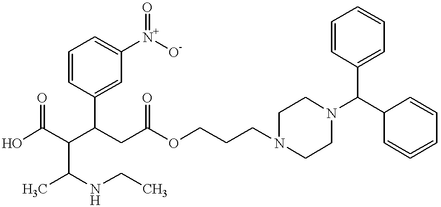

### EXAMPLE 2

The inhibiting activity of the compound obtained in Example 1 on N-type calcium channel and L-type calcium channel was determined. It exhibited the activity of selectively inhibiting N-type calcium channel. Thus, the compounds of the present invention are useful in the treatment of and as remedies for various diseases, such as acute stage of ischemic cerebrovascular disorders caused by cerebral infarction or intracerebral bleeding and Alzheimer's disease.

Having described the present invention, it will be clear to one of ordinary skill in the art that many changes and modifications may be made to the above-described embodiments without departing from the spirit and scope of the present invention.

What is claimed is:

1. A dihydropyridine compound of the following formula (1) or a pharmaceutically acceptable salt thereof: 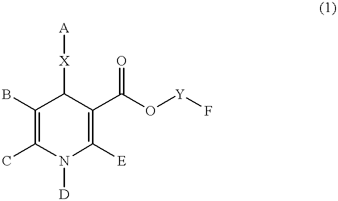
wherein A represents a group of the following formula (2), or 1-naphthyl, 2-naphthyl, thiophene-3-yl, thiophene-2-yl, furan-3-yl, furan-2-yl, pyridine-4-yl, pyridine-3-yl, pyridine-2-yl, indole-2-yl, indole-3-yl, quinoline-2-yl, quinoline-3-yl, quinoline-4-yl, quinoline-5-yl, quinoline-6-yl, quinoline-7-yl, quinoline-8-yl, cyclohexyl or cyclopentyl: 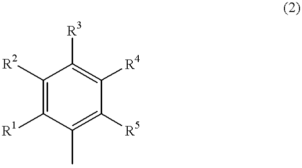
wherein R¹, R², R³, R⁴ and R⁵ is each the same or different from each other and each represent hydrogen, halogen, hydroxyl, carboxyl, amino, cyano, nitro, lower alkyl, lower alkoxyl, lower alkenyl, lower alkynyl, lower alkylamino, lower alkylthio, lower alkanoyl, hydroxy-lower alkyl, hydroxy-lower alkoxyl, hydroxy-lower alkenyl, halogeno-lower alkyl, halogeno-lower alkoxyl, halogeno-lower alkenyl, aryl-lower alkoxyl, lower-alkoxycarbonyl, aryl, heteroaryl, or aroyl;
B represents acetyl or carboxyl;
C represents hydrogen, methyl, ethyl or dimethoxymethyl;
D represents hydrogen, lower alkyl, hydroxy-lower alkyl, or aryl-lower alkyl;
E represents hydrogen, methyl, ethyl, dimethoxymethyl or cyano;
F represents a heterocyclic or cycloalkyl;
X represents an interatomic bond, —CH₂—, —CH₂CH₂—, —CH═CH— or —C═C—, and
Y represents an interatomic bond, —CH₂— or a group of any of the formulae (3) to (15): 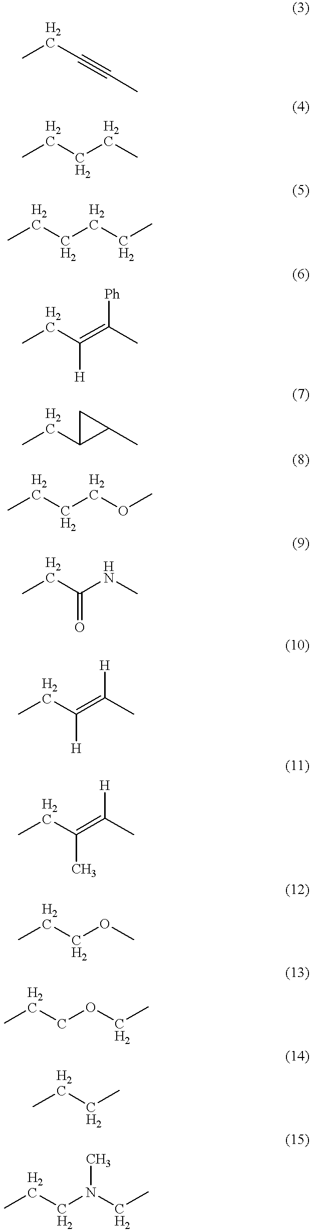
with the proviso that when Y represents any of the groups of the formula (3) to (15), the heterocyclic groups represented by F exclude groups of the following formula (16), cyclohexyl, thiophene-3-yl, thiophene-2-yl, furan-3-yl, furan-2-yl, pyridine-4-yl, pyridine-3-yl and pyridine-2-yl: 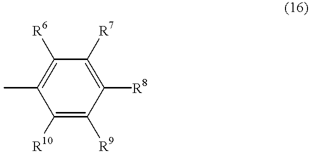
wherein R⁶, R⁷, R⁸, R⁹ and R¹⁰ is each the same or different from each other, and each represent hydrogen, halogen, hydroxyl, carboxyl, amino, cyano, nitro, a lower alkyl, lower alkoxyl, lower alkenyl, lower alkynyl, lower alkylamino, lower alkylthio, lower alkanoyl, hydroxy-lower alkyl, hydroxy-lower alkoxyl, hydroxy-lower alkenyl, halogeno-lower alkyl, a halogeno-lower alkoxyl, halogeno-lower alkenyl, aryl-lower alkoxyl, a lower-alkoxycarbonyl or an aroyl; or
two of R¹ through R³ in the formula (2) are optionally bonded to each other to form a ring.

2. The dihydropyridine compound or pharmaceutically acceptable salt thereof of claim 1, wherein the heterocycle group F comprises pyrrolidine, piperazine, pyrazolidine, imidazolidine, tetrahydrofuran, tetrahydropyran, dioxane, tetrahydrothiophene, morpholine, imidazole, pyrrolidinone, oxazole, isoxazole, pyrimidine, pyrazine, pyridazine or piperidine.

3. The dihydropyridine compound or pharmaceutically acceptable salt thereof of claim 2, wherein R¹, R², R³, R⁴ and R⁵ in the formula (2), which are the same or different from each other, represent hydrogen, halogen, hydroxyl, carboxyl, cyano, nitro, a lower alkyl, lower alkoxyl, halogeno-lower alkyl or lower-alkoxycarbonyl.

4. The dihydropyridine compound or pharmaceutically acceptable salt thereof of claim 3, wherein D represents hydrogen; X represents an interatomic bond; and Y represents an interatomic bond, methylene, ethylene or propylene.

5. The dihydropyridine compound or pharmaceutically acceptable salt thereof of claim 4, wherein B represents carboxyl.

6. The dihydropyridine compound or pharmaceutically acceptable salt thereof of claim 5, wherein R¹, R², R³, R⁴ and R⁵ in the formula (2), which are the same or different from each other, represent hydrogen, halogen, carboxyl, cyano, nitro or halogeno-lower alkyl; C represents methyl; E represents methyl and F represents pyrrolidine, piperazine, imidazole, pyrrolidinone or piperidine.

7. The dihydropyridine compound or pharmaceutically acceptable salt thereof of claim 5, wherein A is represented by the formula (2), wherein R¹, R³, R⁴ and R⁵ each represent hydrogen; and R² represents nitro, C represents methyl, E represents methyl and F represents piperidine or piperazine.

8. The dihydropyridine compound or pharmaceutically acceptable salt thereof of claim 7, wherein B represents carboxyl; D represents hydrogen; X is an interatomic bond; Y represents an interatomic bond, methylene, ethylene or propylene.

9. The dihydropyridine compound or pharmaceutically acceptable salt thereof of claim 1, which is mono(2-(4-benzhydrylpiperazine-1-yl)ethyl) 2,6-dimethyl-4-(3-nitrophenyl)-1,4-dihydropyridine-3,5-dicarboxylate.

10. A N-type calcium channel antagonist composition containing one or more dihydropyridine compounds of the following formula (1) or the pharmaceutically acceptable salts thereof as an active ingredient: 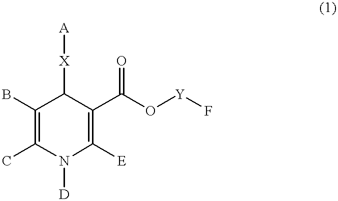
wherein A represents a group of the following formula (2), or 1-naphthyl, 2-naphthyl, thiophene-3-yl, thiophene-2-yl, furan-3-yl, furan-2-yl, pyridine-4-yl, pyridine-3-yl, pyridine-2-yl, indole-2-yl, indole-3-yl, quinoline-2-yl, quinoline-3-yl, quinoline-4-yl, quinoline-5-yl, quinoline-6-yl, quinoline-7-yl, quinoline-8-yl, cyclohexyl or cyclopentyl: 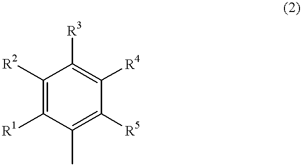
wherein R¹, R², R³, R⁴ and R⁵ are the same or different from each other and each represent hydrogen, halogen, hydroxyl, carboxyl, amino, cyano, nitro, lower alkyl, lower alkoxyl, lower alkenyl, lower alkynyl, lower alkylamino, lower alkylthio, lower alkanoyl, hydroxy-lower alkyl, hydroxy-lower alkoxyl, hydroxy-lower alkenyl, halogeno-lower alkyl, halogeno-lower alkoxyl, halogeno-lower alkenyl, aryl-lower alkoxyl group, lower-alkoxycarbonyl, aryl, heteroaryl or aroyl,
B represents carbamoyl, cyano, nitro, acetyl or carboxyl;
C represents a hydrogen atom, methyl group, ethyl group or dimethoxymethyl group,
D represents hydrogen, lower alky, hydroxy-lower alkyl or aryl-lower alkyl;
E represents hydrogen, methyl, ethyl, dimethoxymethyl or cyano;
F represents heterocyclic or cycloalkyl;
X represents an interatomic bond, —CH₂—, —CH₂CH₂—, —CH═CH— or —C═C—, and
Y represents an interatomic bond, —CH₂— or a group of any of the following formulae (3) to (15): 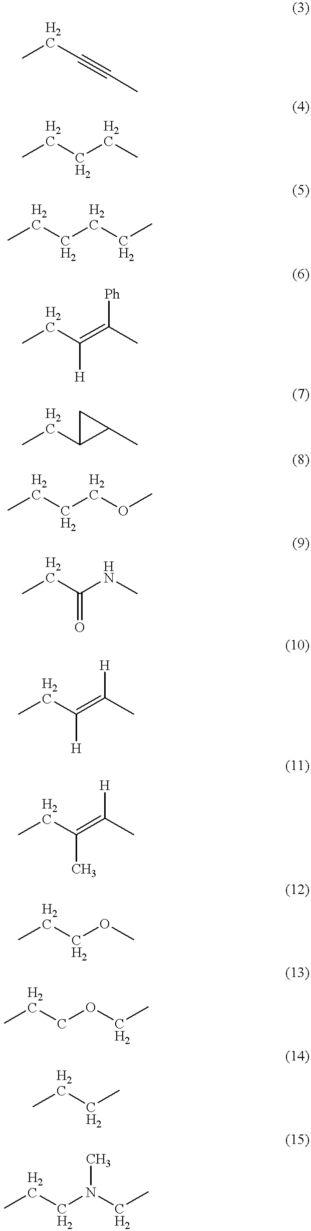
with the proviso that when Y represents any of the groups of the formulae (3) to (15), the heterocyclic groups represented by F exclude groups of the following formula (16), cyclohexyl, thiophene-3-yl, thiophene-2-yl, furan-3-yl, furan-2-yl, pyridine-4-yl, pyridine-3-yl and pyridine-2-yl: 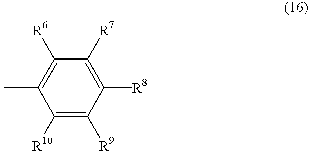
wherein R⁶, R⁷, R⁸, R⁹ and R¹⁰ are the same or different from each other, and each represent hydrogen, halogen, hydroxyl, carboxyl, amino, cyano, nitro, lower alkyl, lower alkoxyl, lower alkenyl, lower alkynyl, lower alkylamino, lower alkylthio, lower alkanoyl, hydroxy-lower alkyl, hydroxy-lower alkoxyl, hydroxy-lower alkenyl, halogeno-lower alkyl, halogeno-lower alkoxyl, halogeno-lower alkenyl, aryl-lower alkoxyl, lower-alkoxycarbonyl or aroyl; and
two of R¹ through R³ in the formula (2) are optionally bonded to each other to form a ring.

11. The N-type calcium channel antagonist composition of claim 10, wherein the heterocycle F comprises pyrrolidine, piperazine, pyrazolidine, imidazolidine, tetrahydrofuran, tetrahydropyran, dioxane, tetrahydrothiophene, morpholine, imidazole, pyrrolidinone, oxazole, isoxazole, pyrimidine, pyrazine, pyridazine or piperidine.

12. The N-type calcium channel antagonist composition of claim 11, wherein R¹, R², R³, R⁴ and R⁵ in formula (2), which are the same or different from each other, represent hydrogen, halogen, hydroxyl, carboxyl, cyano, nitro, lower alkyl, lower alkoxyl, halogeno-lower alkyl or lower-alkoxycarbonyl.

13. The N-type calcium channel antagonist composition of claim 12, wherein D represents hydrogen, X represents an interatomic bond, and Y represents an interatomic bond, methylene, ethylene or propylene.

14. The N-type calcium channel antagonist composition of claim 13, wherein, B represents carboxyl.

15. The N-type calcium channel antagonist composition of claim 14, wherein R¹, R², R³, R⁴ and R⁵ in formula (2), which are be the same or different from each other, represent hydrogen, halogen, carboxyl, cyano, nitro or halogeno-lower alkyl, C represents methyl, E represents methyl and F comprises pyrrolidine, piperazine, imidazole, pyrrolidinone or piperidine.

16. The N-type calcium channel antagonist composition of claim 14, wherein A is represented by the formula (2), wherein R¹, R³, R⁴ and R⁵ each represent hydrogen and R² represents nitro, C represents methyl, E represents methyl, and F represents piperidine or piperazine.

17. A therapeutic composition, comprising one or more of the dihydropyridine compounds or the pharmaceutically acceptable salts thereof of claim 1, as the active ingredient, in an amount effective for treatment of the acute stage of ischemic cerebrovascular disorders caused by cerebral infarction or intracerebral bleeding, Alzheimer's disease, AIDS related dementia, Parkinson's disease, progressive neurodegenerative diseases, dementia due to cerebrovascular disorder, pain caused by thromboangiitis obliterans, postoperative pain, migraine, visceral pain, bronchial asthma, unstable angina, irritable colitis or withdrawal symptoms after addiction to drugs; and a pharmaceutically acceptable carrier.

18. The therapeutic composition of claim 17, wherein the heterocycle F comprises pyrrolidine, piperazine, pyrazolidine, imidazolidine, tetrahydrofuran, tetrahydropyran, dioxane, tetrahydrothiophene, morpholine, imidazole, pyrrolidinone, oxazole, isoxazole, pyrimidine, pyrazine, pyridazine or piperidine.

19. The therapeutic composition of claim 17, which is in a form of tablets, powders, pills, granules, capsules, suppositories, solutions, sugar-coated tablets or depots.

20. The therapeutic composition of claim 17, which is in unit dosage form comprising about 1 μg to 5 g of said one or more dihydropyridine compounds.

21. The therapeutic composition of claim 20, which is in dosage form comprising about 0.01 μg to 1 g of said one or more dihydropyridine compounds.
